(19) United States
(12) Patent Application Publication
(10) Pub. No.: US 2022/0313225 A1
(43) Pub. Date: Oct. 6, 2022

(54) ENCLOSURE SYSTEM AND PROCESS FOR REMOVAL OF UNDESIRED CELLS FROM THE HUMAN BODY

(71) Applicant: OROC, Bay Village, OH (US)

(72) Inventor: Saubhik SENGUPTA, Kolkata (IN)

(73) Assignee: OROC, Bay Village, OH (US)

(21) Appl. No.: 17/710,897

(22) Filed: Mar. 31, 2022

Related U.S. Application Data
(60) Provisional application No. 63/168,809, filed on Mar. 31, 2021.

(51) Int. Cl. A61B 10/00; A61M 37/00

(52) U.S. Cl. CPC A61B 10/0045; A61M 37/00

(57) ABSTRACT

An enclosure system for removal of undesired cells from a human body that contains a tumor formed of the undesired cells includes an enclosure body that is elastic and expandable, formed with a cavity and a closable opening, and includes at least a first port and a second port. The body is constructed for placement in a desired location of the human body near the tumor, and is capable of receiving a first set of chemicals through port A and a second set of chemicals through port B. The second set of chemicals includes a preselected chemoattractant that is operable to direct the undesired cancer cells, including the tumor, toward them. The body is constructed to enclose the tumor and associated cancer cells that move into the cavity because of the chemoattractant, to be closed after a preselected time period and, thereafter, to be removed from the human body.

### CROSS-REFERENCES TO RELATED APPLICATION

The present application claims the benefit under 35 U.S.C. § 119(e) of U.S. Provisional Patent Application Ser. No. 63/168,809, filed Mar. 31, 2021 and entitled “POUCH BAG SYSTEM AND PROCESS FOR REMOVAL OF CANCER CELLS FROM THE HUMAN BODY”, the disclose which is incorporated herein by reference.

### SUMMARY OF THE INVENTION

The present invention relates to an enclosure system and process for removal of undesired cells from the human body. One version is a pouch bag system and process for removal of cancer cells from the human body related to naïve, metastatic and recurrent solid tumor cells.

As described below in connection with FIG. 1, the invention includes an enclosure system for removal of undesired cells from a human body that contains a tumor formed of the undesired cells. That system includes an enclosure body that is elastic and expandable, formed with a cavity and a closable opening, and includes at least a first port and a second port. The body is constructed for placement in a desired location of the human body near the tumor, and is capable of receiving a first set of chemicals through port A and a second set of chemicals through port B. The second set of chemicals includes a preselected chemoattractant that is operable to direct the undesired cancer cells, including the tumor, toward them. The body is constructed to enclose the tumor and associated cancer cells that move into the cavity because of the chemoattractant, to be closed after a preselected time period and, thereafter, to be removed from the human body.

Chemoattractants are chemicals that direct the movement of cancer cells in the body leading to metastasis.

In case of ovarian cancer, 18:1 lysophosphatidic acid (LPA) has been shown to be the molecule that is released by ovarian cancer cells and mesothelial cells, allowing for the cells to metastasize to different parts of the body.

In other solid tumor oncologies, the chemoattractant leading to metastasis of cells varies from tumor to tumor (e.g., epidermal-growth-factor receptor (EGF) can lead to metastasis of breast cancer cells). Chemoattractants work by stimulating cancer-cell-surface receptors and which gives them a direction to metastasize.

The present invention uses the directional nature of chemoattractants to direct cancer cells toward and into an enclosure, such as a pouch bag, that has been implanted in a human body in a desired location. Then, after a preselected amount of time, the cancer-cell-containing enclosure is removed from the human body. Accordingly, the enclosure system and the process of using it, can remove tumor cells from a human body without giving the toxicities of chemotherapy or the morbidities of surgery.

Chemoport is a conventional medical device used in a surgical procedure for cancer patients requiring chemotherapy. The pouch bag is guided through the chemoport using conventional catheterization techniques to reach the nearest point of a solid tumor in the cancer-containing human body. The pouch bag will contain the defined chemoattractant (in case of ovarian cancer it will be LPA and for other tumors the chemoattractant will be defined using expression data of the plasma membrane protein receptors obtained by RNA seq NGS.

One version of the enclosure system and process of the present invention is a pouch bag system and method that contains a preselected chemoattractant to direct cancer cells to move toward, and metastasize in, the pouch bag. The bag is placed near the tumor site through the conventional Chemoport catheratization technique. Once the tumor cells have move toward and metastasized in the bag, the bag is removed from the human body through the Chemoport.

### BRIEF DESCRIPTION OF THE DRAWING

FIG. 1 is a schematic view of the enclosure system and method of the present invention that shows the pouch-bag version of it.

### DETAILED DESCRIPTION

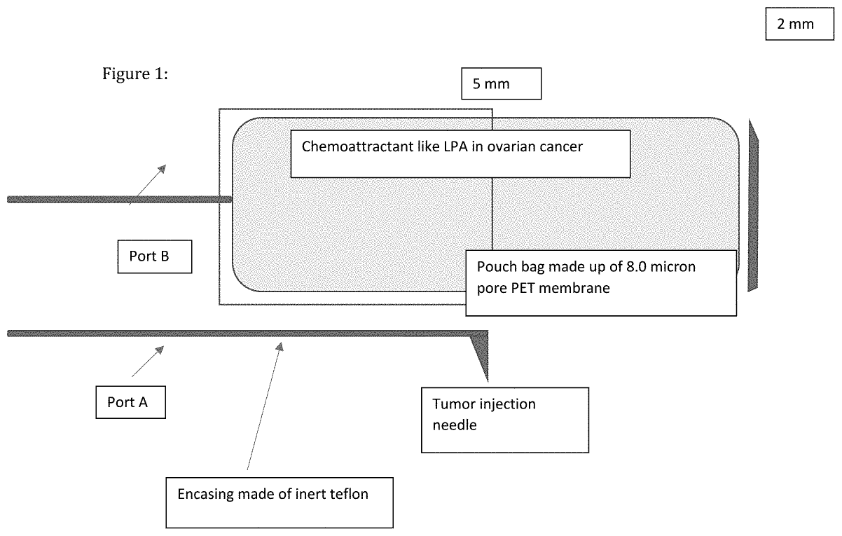

Referring to FIG. 1, the following description explains the pouch-bag version of the invention:

1. A cancer patient is diagnosed with solid tumor oncology, or the patient had been diagnosed, treated earlier, and had recurred with the disease.

2. CT scan or whole body PET scan reveals the site of a metastasis.

3. A CT-guided or USG-guided biopsy is performed and RNA seq of plasma membrane proteins is performed to identify the desired chemoattractant target.

4. If Chemoport is already surgically implanted then Chemoport is identified. If Chemoport is not implanted, then the Chemoport is implanted.

5. A pouch bag with an opening and ports A and B is inserted through the Chemoport under radiological surveillance to place it near the metastasis (tumor).

6. Collagenase I and DNAse I are injected through port A of the pouch bag to digest the cell-cell adhesion contact of the cells in the tumor.

7. After 1 hour, normal saline is injected through port A of the pouch bag for washing of the enzymes.

8. After 1 hour, the desired chemoattractant is injected through port B of the pouch bag. The pouch bag will now expand because of the liquid.

9. The Chemoport is closed, and the pouch bag remains in the cancer patient's body for 48 hours.

10. After 48 hours, the pouch bag is removed through the Chemoport.

11. A CT scan or PET CT scan is performed to confirm that the tumor has been removed.

12. For multiple metastatic nodules, the process is repeated.

What is claimed:

1. An enclosure system for removal of undesired cells from a human body that contains a tumor formed of the undesired cells, comprising:
an enclosure body that is elastic and expandable, formed with a cavity and a closable opening, and includes at least a first port and a second port;
wherein, the body is constructed for placement in a desired location of the human body near the tumor, and capable of receiving a first set of chemicals through port A and a second set of chemicals through port B;
wherein the second set of chemicals includes a preselected chemoattractant that is operable to direct the undesired cancer cells, including the tumor, toward them; and
wherein the body is constructed to enclose the tumor and associated cancer cells that move into the cavity because of the chemoattractant, to be closed after a preselected time period and, thereafter, to be removed from the human body.

2. The system of claim 1, wherein the body is formed as a pouch bag.
